(19) United States
(12) Patent Application Publication
(10) Pub. No.: US 2013/0146064 A1
(43) Pub. Date: Jun. 13, 2013

(54) Nasal Cannula Support Device

(71) Applicant: Helen Dryden, Casper, WY (US)

(72) Inventor: Helen Dryden, Casper, WY (US)

(21) Appl. No.: 13/709,099

(22) Filed: Dec. 10, 2012

Related U.S. Application Data
(60) Provisional application No. 61/568,303, filed on Dec. 8, 2011.

(51) Int. Cl. A61M 15/08

(52) U.S. Cl. CPC A61M 15/08
USPC 128/207.18

(57) ABSTRACT

A harness for securing a nasal cannula to a user for the purpose of delivering oxygen. The harness comprises an upper and lower strap located at the top and rear of a user's head, respectively. The upper and lower straps are orthogonally connected at their midpoint by a vertical connecting strap, and contain on either end, an adapted means to secure and support a nasal cannula by looping around the plastic tubing and then attaching onto itself. Two distinct connection points above and below each ear maintain the suspended position of the cannula without deforming its cross section. By providing a three-strap methodology for supporting the nasal cannula and maintaining positive control of the plastic tubing by keeping it suspended above the ears, the present invention avoids commonly associated drawbacks related to existing devices.

### CROSS REFERENCE TO RELATED APPLICATION

This application claims the benefit of U.S. Provisional Application No. 61/568,303 filed on Dec. 8, 2011 entitled “Oxytight.” The patent application identified above is incorporated here by reference in its entirety to provide continuity of disclosure.

### BACKGROUND OF THE INVENTION

1. Field of the Invention

The present invention relates to oxygen delivery devices and supports therefor. More specifically, the present invention pertains to a nasal cannula support harness that securely fastens to four discrete locations along the cannula tubing for securing the assembly to a user in order to prevent movement while routing the tubing in such a manner that reduces cannula dislodgement and skin irritation.

An oxygen delivery system is critical for many people who suffer from forms of lung disease or lack the physical capacity to adequately supply oxygen to themselves. Oxygen therapy works to alleviate strain placed on the body by supplying the necessary amount of oxygen needed for cell metabolism and tissue oxygenation. During therapy the communicated air is generally pressurized and introduced directly into the user's nasal or oral airways, while the air can further be enriched to maximize oxygen absorption.

A common means of implementing oxygen therapy is utilizing a pressurized oxygen source and a nasal cannula. A nasal cannula is a device comprising flexible plastic tubing and a small nasal prong assembly that is placed into the user's nasal passage for communicating air thereinto. The tubing emanating from the oxygen source divides into two branches below the chin, is routed to opposite sides of a user's head along the jawbone, looped around the top of the ears and then extended along the user's cheeks before arriving at the nostrils. The nasal prong assembly comprises two small prongs or tubes that protrude into the nostrils of a user and directly pump oxygen into the user's respiratory system.

Cannula devices are extensively utilized in the healthcare field for oxygen therapy; however their design has inherent drawbacks that limit their use and their effectiveness with regard to user comfort and therapy compliance. The first of these drawbacks relates to discomfort and skin irritation that results from movement of the cannula with respect to the user's skin over a period of time. This movement creates friction against the skin and can quickly lead to rashes, sores and even infections along the affected areas. A second major drawback relates to the propensity of the cannula nasal prongs to dislodge from the user's nasal passages during head movement or while the user is sleeping. Since the cannula is generally supported only over the ears of the user, wherein the nasal prongs rest in the user's nose the prongs, maintenance of the cannula position is achieved only through tension along the length of the cannula and tightening thereof around the back of the user's ears, which is a cause of the aforementioned skin irritation.

Of primary concern for the present invention is the ability to securely fasten a nasal cannula to a user while not causing discomfort. Predominately, people who utilize oxygen therapy are in a weakened state and may not have the energy to constantly reposition their equipment in order to receive the prescribed treatment. It is also prudent to consider users who are on oxygen therapy over an extended period of time and have a portable oxygen tank constantly in tow. This creates numerous opportunities for the tubing to be snagged or caught on objects that exist in their path of transport. The moment the plastic tubing is caught, the nasal prongs that protrude into the nostrils may be either ejected or pushed forward, which undoubtedly causes discomfort for a user. This issue can be directly linked to the absence of a structural support for the cannula device when deployed.

Support for the cannula may also be required during periods of sleep, when a user may dislodge the nasal cannula due to unconscious movement, such as tossing and turning. Besides interrupting a user's regular sleeping pattern, this behavior can have a negative impact on position of the cannula and thus negate the positive effects of the oxygen therapy. Devices exist in the prior art that relate to cannula support devices, however these do not address this issue while proving comfort and stability, as is provided by the present invention. By providing the necessary support and a comfortably fitting cannula support, a user is less likely to be constantly aware of the presence of the support, thus the user will be less likely to circumvent a treatment, whether it be by their own will or not, thereby providing a better means for obtaining a healthier lifestyle and reducing pain associated with unsupported or improperly supported cannulas.

2. Description of the Prior Art

The present invention addresses the prominent drawbacks relating to nasal cannula support devices that currently reside in the art. The device is comprised of two main components, a means to secure to the cannula at discrete points along its length using straps, and a harness to comfortably attach the device to a user. Disclosed are four total connection points made between the plastic tubing and the harness, two of which are located above each ear and two being therebehind. At the attachment points, a strap extending from the harness is looped around the tubing and then back onto itself, thereby providing sufficient support that prevents movement of the nasal cannula, while not restricting the flow of oxygen by contracting the diameter thereof. By keeping the tubing securely suspended above the ear, the present invention maintains the nasal prong positions via tension along the cannula, while not creating a protruded harness or headgear along the rear of the head that would otherwise cause discomfort in the supine position. The harness section is comprised of an upper and lower strap that contains a means to secure the plastic tubing on each end, wherein the assembly is designed to comfortably engage and support the cannula tube above the user's ears without requiring the user to wear bulky or readily dislodgable structures. The goal is to provide a support that the user can readily forget and one that comfortably and stably supports the cannula in a working position.

Several nasal cannula support devices have been previously disclosed in the prior art. These include devices that have been patented and published in patent application publications. One such device is U.S. Pat. No. 6,536,436 to McGlothen, which describes a tube holding system. This device describes how tubing is routed from a user's nasal passage, around each ear and then to the back of the neck where both tubes are secured using a strap having hook and loop fasteners. By relying on the tubing to be in constant contact with the skin, pinch points can occur on both on the upper ear lobe as well as the temporal region if tension on the tubing is applied in a downward or horizontal direction, respectively. These pinch points cause undo friction that can lead to chaffing, sores, rashes and infections in a user. The stability of this device when attached to a user is also insufficient due to having only one strap that secures the plastic tubing along the nape of the neck. The location of this lone strap will cause a problem in users endowed with hair that extends past their shoulders since the mated material will undoubtedly catch the hair follicles causing undue pain and discomfort. The present device negates these hazards by keeping the tubing suspended above the ear with hook and loop fasteners and provides a secure attachment with three straps and additional webbing encircling the perimeter of the skull.

U.S. Pat. No. 6,899,102 to McGlothen is another device that describes a method of placing a plastic tube holding device on a user. The system places the plastic tubing around the ear lobe and utilizes hook and loop fasteners to secure the tubing within one inch behind and below each ear lobe. The strap should in no way extend above the earlobes, which could result in an interference regarding the maneuverability of the user. This method provides a secure means to attach the tubing yet will cause discomfort to the top of the ear lobe as well as the temporal region of the user due to contact between the plastic tubing and skin.

Further, U.S. Pat. No. 4,836,200 to Clark discloses an oxygen delivery system comprising a single strap that utilizes two loops to maintain the suspension of the plastic tubing above the ears. This approach negates the skin irritation issue that is commonly associated with current devices in the art by successfully eliminating constant contact between the plastic tubing and a user's skin. While providing a feasible approach to end the skin irritation issue, a one-strap approach does not prevent movement of the tubing during transit of a user. Any rotation of the skull will undoubtedly shift the tubing out of place, which requires a user to constantly readjust this device causing discomfort and frustration. A three-strap design adds substantially more support against movement and provides a more secure fastening method.

Finally, U.S. Pat. No. 4,665,566 to Garrow comprises a strap that secures plastic tubing to a user via Velcro fasteners. The strap is intended to wrap around the perimeter of the skull, or other extremity, and then be tightened at the discretion of a user. Once the strap is situated, the tubing is routed through an opening in the strap on one side relative to a user, then to the nasal passage and back to the strap on the side opposite the initial route in order to form a closed loop. The tubing is then secured by folding the Velcro fasteners upon themselves, thus creating enough pressure to hold the device in place. While the tubing is secure in this fashion, a problem can arise in establishing an efficient level of tension between the nose and skull strap, which can cause irritation in nasal bridge. If this device is worn for a long period of time the pressure required to keep the device in place along the forehead is another means for discomfort to a user. Also, while a user is in the supine position, their head would rest on an unlevel surface resulting from the protruding strap which will undoubtedly cause neck and head pain. The present device solves these issues by not contributing any bulky protrusions for a user to come in contact with while in the supine position and offers a harness methodology that will not add unnecessary pressure in the form of tension thereby eliminating discomfort.

The present invention differs significantly in structure and spirit from the prior art devices. These prior art devices disclose a novel means for deploying a nasal cannula support apparatus, yet in some manner fail to completely address stability concerns and skin irritation issues commonly found in the field while maintaining an unobtrusive presence. The present invention creates a novel solution to these problems by providing a three-strap harness and four points of attachment to the nasal cannula. The three-strap harness ensures a stable connection when deployed due to multiple areas of contact between the harness and a user's head. The four points of attachment to the nasal cannula further facilitate the structural integrity of the invention while keeping the tubing suspended above the ear thus negating skin irritation for a user. The structure and construction of the present device further reduces constant awareness of the device and interference from the device while positioned on a user's head. It is therefore submitted that the disclosed device substantially diverges in design elements from the prior art and consequently it is clear that there is a need in the art for an improvement to existing nasal cannula support devices. In this regard the instant invention substantially fulfills these needs.

### SUMMARY OF THE INVENTION

In view of the foregoing disadvantages inherent in the known types of nasal cannula support devices now present in the prior art, the present invention provides a new nasal cannula support device wherein the same can be utilized for providing convenience for a user when undergoing oxygen therapy and securely supporting the cannula within the nasal passages of a user using a comfortable and readily deployable structure.

It is therefore an object of the present invention to provide a new and improved nasal cannula support device that has all of the advantages of the prior art and none of the disadvantages.

It is another object of the present invention is to provide a nasal cannula support device that eliminates irritation for a user in the form of chaffing, rashes or sores by means of contact between the plastic tubing and the skin.

Another object of the present invention is to provide a nasal cannula support device that negates irritation for a user in the form of pinch points. Pinch points are formed when the tension used to secure the nasal cannula is transferred to a user via the ear lobe, temporal region, nostril or nape of the neck.

Another object of the present invention is to provide a nasal cannula support device that utilizes thin fabric construction and a rear head-mounted harness that is not bulky and does not interfere with the user while sleeping or during daily activities.

Another object of the present invention is to provide a nasal cannula support device that is firmly seated against a user's head via a three-strap methodology and optional comfort webbing that emanates from a center strap, thereby transferring motion of the cannula to the harness to prevent movement and creating a comfortable structure to be worn by the user.

Yet another object of the present invention is to provide a nasal cannula support device that does not protrude from a user's skull in such a way as to cause discomfort while in the standing, sitting or supine positions.

A final object of the present invention is to provide a nasal cannula support device that prevents displacement of the nasal prongs during transportation or movement by applying tension along the cannula to maintain its connection with the nostrils, while supporting the cannula above the ears of the user for reduced skin irritation.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the present invention in a working state, wherein the harness that supports the plastic tubing is visualized.

FIG. 2 shows a perspective close-up view of the present invention in a working state from behind a user's ear, wherein a first and second attachment point along the cannula length secure tubing around the perimeter of the ear.

FIG. 3 shows a perspective view of the present invention in a working state, wherein the upper and lower straps, as well as the strap material that links them, are visualized from the rear of the skull.

FIG. 4 shows a perspective view of the present invention while not in a working manner.

FIG. 5 shows an embodiment of the present invention while not in a working manner.

### DETAILED DESCRIPTION OF THE INVENTION

Reference is made herein to the attached drawings. Like reference numerals are used throughout the drawings to depict like or similar elements of the nasal cannula support device. For the purposes of presenting a brief and clear description of the present invention, the preferred embodiment will be discussed as used for securing a nasal cannula device to a user for the purpose of comfortably and securely delivering oxygen via a nasal cannula. The figures are intended for representative purposes only and should not be considered to be limiting in any respect.

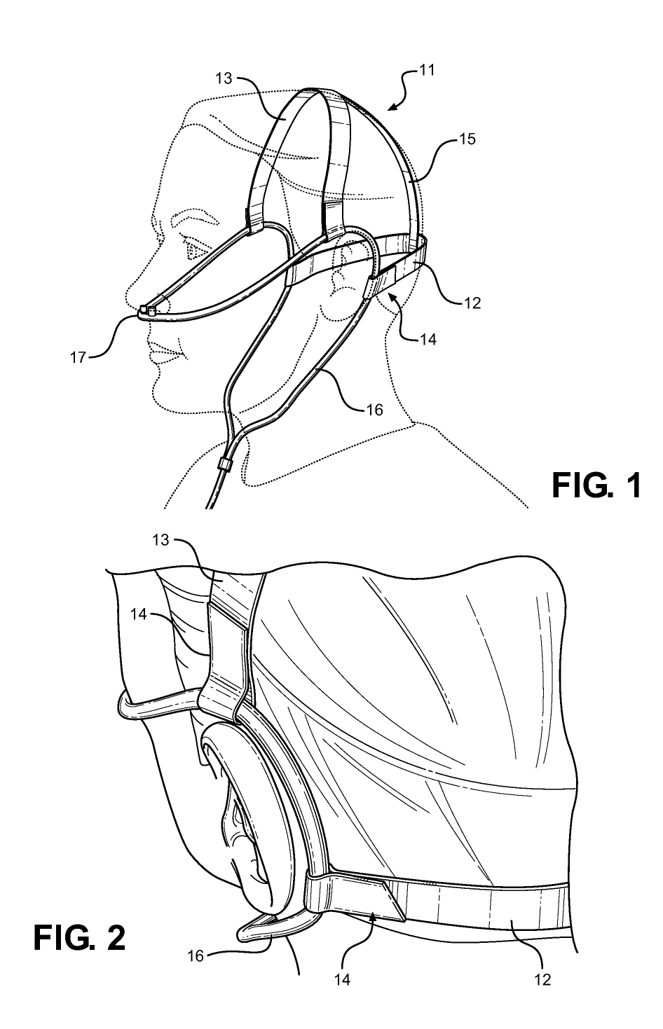

Referring now to FIG. 1, there is shown a perspective view of the nasal cannula support device 11 in a working manner as described in the present invention. The device comprises a harness that cradles the rear of a user's head using a first, send and third that creates four cannula attachment points to provide stability and support thereto. An upper strap 13 is symmetrically deployed across a user's head from above the ear and attaches to a lower strap 12 via an orthogonal center strap 15 running along the middle of a user's head, wherein each strap shares similar material, width and thickness. The lower strap 12 begins behind each ear and is routed across the back of the head. At the terminal ends of the upper and lower straps there exists a means to secure the cannula tubing to each end while keeping the tubing 16 suspended above and around the perimeter of the ear without pinching the cannula cross section. The strap attachment points are located above and behind the ears and each secure to the nasal cannula by looping around the plastic tubing 16 and then mating back onto itself. Left unsupported, movement by a user would dislodge the plastic tubing 16 and the nasal prongs 17, which would necessitate constant repositioning and tightening of the cannula around the back of each ear, increasing skin contact, rubbing and therefore irritation. By utilizing a harness design that is adapted to support the cannula from the back of the wearer's head using three different straps and four attachment points, this type of inconvenience is negated. Further, the use of thinly sectioned straps reduces the cross section of the harness, thereby allowing its presence to go relatively unnoticed and be non-intrusive on the user's daily activities, including when resting in a supine position.

The means for securing the terminal ends of each strap to the cannula tubing can be accomplished with an assortment of contemplated methods, including hook and loop fasteners, snaps, buckles or laces. Although hook and loop fasteners 14 are shown in the present embodiment the device, it is not desired to limit this connection to a specific type of fastening means, wherein one or more efficient means may be available. The ability of each strap to loop around the cannula cross section and secure against itself in two discrete locations around each ear reduces pinch points and increases the stability of the cannula. The straps that comprise the harness can be suitably made from a variety of materials, including elastic cotton, polyester, or neoprene, wherein it is desired to limit their thickness and broaden their width to facilitate their ability to secure the cannula without creating a bulky headgear device. The use of a soft and flexible material permits the device to be worn on the head in a comfortable and secure manner while keeping the plastic tubes and nasal prongs in place.

Referring now to FIG. 2, there is shown an enlarged perspective view of the present invention keeping the plastic tubing 16 suspended around the perimeter of the ear. The upper strap 13 is routed in a vertical direction relative to the ear, while the lower strap 12 is traveling horizontally. Both straps supply support to the nasal cannula via their attachment locations and apply tension to the cannula tube to maintain the nasal prong connectivity within the nose and to prevent the back of each ear from supporting any section of the tubing. Portions of each straps loop around the plastic tubing and then back onto themselves to form a secure support for applying tension on the cannula, wherein the tension is supported by the harness connection along the back of the user's head and on either end via connection to the cannula around both ears. The area of contact between the tubing and strap contains a pressure that securely attaches the nasal cannula to a user while not contracting the diameter of the tubing 16, which would result in diminished airflow. The width of the straps creates an elongated area of support that resists pinching the tube along its length, which would otherwise create blockages along the cannula interior that would hinder airflow to the user.

Users requiring a nasal cannula device often route the plastic tubing 16 behind their ears and then let it run along their cheeks. This configuration applies pressure to a user's skin behind each ear, as this area acts as a support surface or bearing support for the cannula, and therefore leads to irritation or inadequate support. These problems may cause a user to wear the tubes less than they should, which can be detrimental to their health. The present invention is therefore disclosed as a means to securely attach the tubing around and above each ear to prevent any contact between the tubing and skin in this region. The present invention is further submitted to provide a support harness that is not intrusive or bulky for the user to regularly wear.

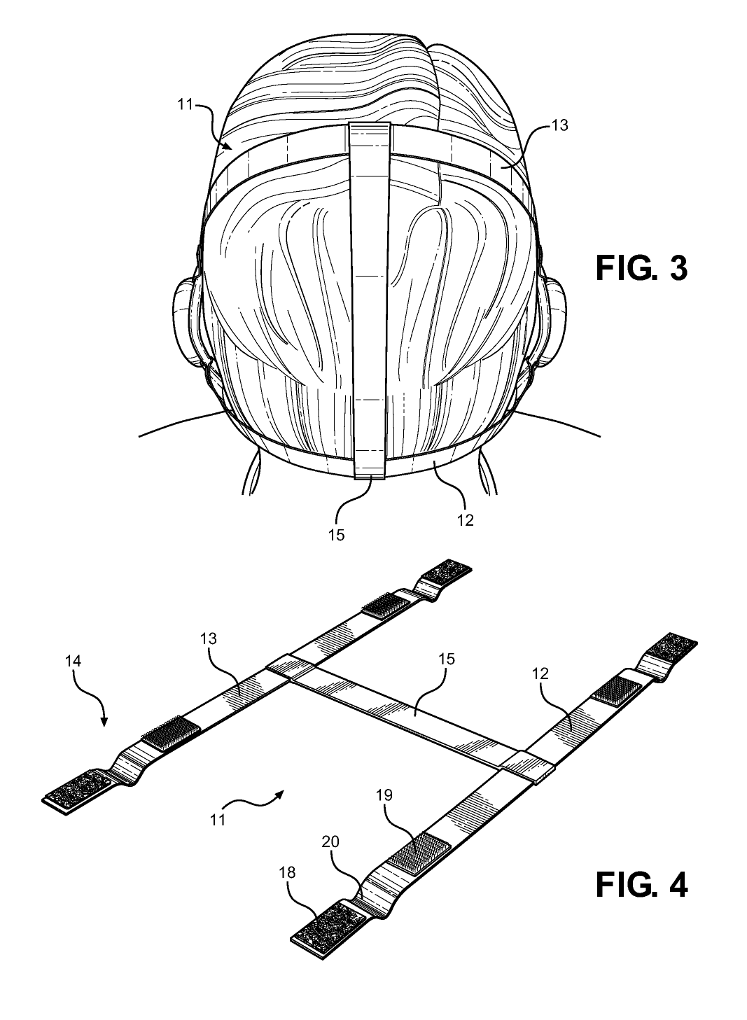

Referring now to FIG. 3, there is shown a rear view of the nasal cannula support device 11 in a working manner. In the preferred embodiment, the harness comprises an upper strap 13 and a lower strap 12 that are orthogonally connected by a central strap 15 of similar material and of uniform width in order to form an I-shaped structure. This configuration gently cradles the top, bottom and rear of the head, thereby securing the nasal cannula and alleviating pressure against the back of both ears, which is the main catalyst for irritation. It has been suggested in the prior art to utilize a one strap device for support, which in comparison to the present invention is prone to displacement due to an inadequate and unsecure attachment. By including a harness design having multiple areas of support for the cannula and widened contact against the back of the user's head, the present invention will create a more secure fit that will inhibit movement of the nasal cannula and increase user comfort while the harness is donned.

Referring now to FIG. 4, there is shown a perspective view of the present invention 11 in an open configuration. The upper strap 13, lower strap 12 and their respective securement means 14 are displayed prior to being deployed. The upper and lower straps are laid out in parallel, having equal lengths and widths, and comprising a two-part, looping connection means at each strap end. At the midpoint of these straps there is a center strap 15 that connects them orthogonally. In a preferred embodiment, the terminal ends 14 of each strap comprise a matching strip of hook 19 and loop 18 fastening material spaced a distance 20 apart to allow each strap to be folded onto itself and around the cannula tube perimeter and secured to itself. However, any looped means of connection that allows the straps to be looped and secured about the cannula is contemplated, wherein the straps can be easily applied and removed from a cannula, and further the harness straps can be adjusted to increase or decrease tension on the cannula utilizing the looped free length of strap. A further embodiment of the present invention includes strap length adjusters along each strap that allow the strap ends to be looped around the cannula and then fed into the adjuster to reduce the length of each strap and thus the tension applied to the cannula by each strap.

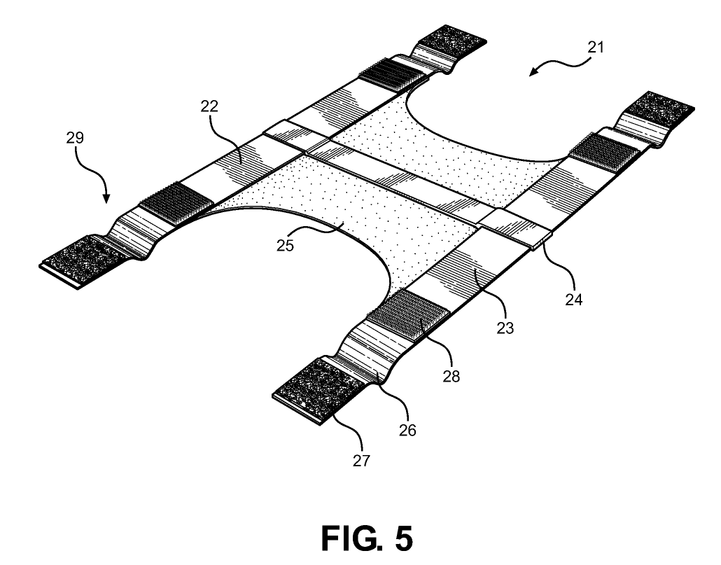

Referring to FIG. 5, there is shown an embodiment of the present invention 21 displayed in an open configuration. This embodiment retains the three-strap structure but incorporates improvements made to increase overall stability and comfort of the invention. The upper 22 and lower 23 straps are laid out in parallel, wherein the midpoint of the straps are connected by a center strap 24. Emanating outward from the center strap is a surface of elastic webbing material 25 that forms two mirrored C-shaped structures wherein the curved edges are facing outboard thus improving the ergonomic fitment of the harness and eliminating any possible interference with the ears of a user. This material is attached to the interior edge of the upper and lower straps and extends outward from the center strap to increase the overall contact area of the harness with the user's head and thus improve comfort and securement thereof. The straps operate as intended in the preferred embodiment, however the webbing 25 supplies increased tension on the straps and reduces discrete areas of contact generated by the use of the straps by themselves. This reduces discomfort along the rear of the user's head and improves the capability to apply tension on the cannula while in operation.

To install the device on a user, the plastic tubing 16 and nasal prongs 17 are moved into position while the harness straps are loosely applied to the cannula or disconnect therefrom. If disconnected, the straps are connected individually to the cannula and then the central strap is placed along the back of the user's head prior to tightening each strap individually. If loosely applied, the same tightening procedure is followed without needing to individually affix each strap. Ideal placement of the harness strap connections along the cannula include above and behind each ear, providing tension that lifts the cannula above the ears to prevent bothersome contact and irritation. Ideal tension on the cannula is such that head movement does not readily dislodge the nasal prongs, while also not unduly forcing the cannula against the user's face and cheeks.

In use, the device provides a novel means of supporting a nasal cannula for the purposes of delivering oxygen and maintaining patient compliance. The device improves upon the prior art by providing an effective means for receiving oxygen therapy in conjunction with a nasal cannula without having to sacrifice either comfort or stability. The three-strap methodology, along with the webbing material, supply multiple areas of contact for attaching the harness to a user thereby negating movement of the plastic tubing and nasal prongs while being deployed. Four attachment points keep the plastic tubing suspended above the ear and are responsible for preventing contact between the nasal cannula and skin, which has a direct correlation to chaffing, sores, rashes and infections. Overall, the present invention provides a drastically improved system that would benefit users in need of oxygen therapy.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A nasal cannula support device, comprising:
an upper and lower strap orthogonally joined by a central connecting strap to form an I-shaped structure, said upper and lower strap each having a first and second terminal end;
said upper, lower and vertical connecting straps having an interior and exterior surface, said interior surface adapted to maintain contact with the back of a user's head;
said upper and lower strap terminal ends having a means of connection whereby said terminal end is looped and removably connected along its respective strap length;
said upper strap and said lower strap termination adapted to connect around a nasal cannula above and behind each ear, respectively and apply tension to said cannula to support said cannula above said ear.

2. The device of claim 1, further comprising a webbing surface between said upper and lower strap.

3. The device of claim 2, wherein said webbing surface is elastic.

4. The device of claim 2, wherein said webbing surface further comprises a curved edge extending between said upper and lower strap for increased ergonomics and reduced ear interference.

5. The device of claim 1, wherein said upper and lower strap of sufficient width to prevent pinching of said cannula cross section at said strap looped connection with said cannula.

6. The device of claim 1, wherein said upper and lower strap further comprises length adjusters to reduce said upper and lower strap length and adjust said cannula tension.

7. The device of claim 1, wherein said upper, lower and central strap are of minimal thickness to allow comfortable supine resting without creating a thickened headgear.

8. The device of claim 1, wherein said straps are fabricated from non-allergenic, soft and flexible material such as rubber, cotton, polyester, or neoprene.

9. A nasal cannula support device, comprising:
an upper and lower strap orthogonally joined by a central connecting strap to form an I-shaped structure, said upper and lower strap each having a first and second terminal end;
said upper, lower and vertical connecting straps having an interior and exterior surface, said interior surface adapted to maintain contact with the back of a user's head;
said upper and lower strap terminal ends having a means of connection whereby said terminal end is looped and removably connected along its respective strap length;
said upper strap and said lower strap termination adapted to connect around a nasal cannula above and behind each ear, respectively and apply tension to said cannula to support said cannula above said ear;
a webbing surface between said upper and lower strap.

10. The device of claim 9, wherein said webbing surface is elastic.

11. The device of claim 9, wherein said webbing surface further comprises a curved edge extending between said upper and lower strap for increased ergonomics and reduced ear interference.

12. The device of claim 9, wherein said upper and lower strap of sufficient width to prevent pinching of said cannula cross section at said strap looped connection with said cannula.

13. The device of claim 9, wherein said upper and lower strap further comprises length adjusters to reduce said upper and lower strap length and adjust said cannula tension.

14. The device of claim 9, wherein said upper, lower and central strap are of minimal thickness to allow comfortable supine resting without creating a thickened headgear.

15. The device of claim 9, wherein said straps are fabricated from non-allergenic, soft and flexible material such as rubber, cotton, polyester, or neoprene.
